(19) United States
(12) Patent Application Publication
(10) Pub. No.: US 2005/0186606 A1
(43) Pub. Date: Aug. 25, 2005

(54) Methods and compositions for detecting nucleic acids

(76) Inventor: Benjamin G. Schroeder, San Mateo, CA (US)

Correspondence Address: HARNESS, DICKEY & PIERCE, P.L.C., P.O. BOX 828, BLOOMFIELD HILLS, MI, 48303, US

(21) Appl. No.: 11/055,849

(22) Filed: Feb. 11, 2005

Related U.S. Application Data
(60) Provisional application No. 60/543,838, filed on Feb. 11, 2004.

(51) Int. Cl. C12Q 1/68; C12N 9/22

(52) U.S. Cl. 435/6; 435/199

(57) ABSTRACT

A reporter molecule for detecting a nucleic acid is disclosed. The molecule comprises an enzyme having a k_{cat} of at least about 200 sec⁻¹, a reversible inhibitor of the enzyme inhibitorily engaging the enzyme, and a nucleobase polymer extending between the enzyme and the inhibitor. The polymer interferes with the engagement between the inhibitor and the enzyme when the nucleic acid contacts the polymer. Methods of making and methods of using the reporter molecule are also disclosed.

### CROSS-REFERENCE TO RELATED APPLICATIONS

This application claims the benefit of U.S. Provisional Application No. 60/543,838, filed on Feb. 11, 2004. The disclosure of the above application is incorporated herein by reference.

### FIELD

The present application relates to nucleic acid detection and, in particular, to methods and compositions for detecting a nucleic acid of known sequence in a sample.

### BACKGROUND

A fundamental aspect of most molecular biology studies involves the detection of nucleic acid sequences. Currently available detection methods generally involve hybridization between a target nucleic acid and a probe complementary to the target. Examples of such methods include blotting methods, such as Southern and Northern blotting (Sambrook et al., Sambrook, J., Fritsch, E. F. and Maniatis, T. (1989) Molecular Cloning: A Laboratory Manual, 2nd ed. Cold Spring Harbor Laboratory Press, Cold Spring Harbor, N.Y.); exonuclease-based methods such as Taqman® assays (Heid, C. A., et al., Genome Res. 6: 986-994, 1996); endonuclease-based methods such as Invader® assays (Lyamichev, V. et al., Nat. Biotechnol. 17: 292-296, 1999), and enzyme activation assays, such as the assay described by Saghatelian et al., Journal of the American Chemical Society 125: 344-345, 2003.

### SUMMARY

The inventor herein has succeeded in devising a new approach for detecting nucleic acids. The approach can be based upon use of a construct that can comprise a nucleobase polymer that is attached to an enzyme and an inhibitor of the enzyme. The conformation of the nucleobase polymer between the attachment sites of the enzyme and inhibitor allows the inhibitor to attach to the enzyme and exert an inhibitory effect on the enzyme in the absence of the target nucleic acid to be detected. In the presence of the target nucleic acid, however, the nucleic acid binds to the nucleopolymer and changes the conformation of the nucleopolymer such that inhibitory attachment of the inhibitor to the enzyme is disfavored. Detection of an increase in measurable amount of enzyme activity (compared to a control) can, therefore, indicate that the nucleic acid is present.

Thus in various embodiments, the present invention can relate to a reporter molecule for detecting a nucleic acid. The reporter molecule can comprise an enzyme having a k_{cat} of at least about 200 sec⁻¹, a reversible inhibitor of the enzyme inhibitorily engaging the enzyme, and a nucleobase polymer extending between the enzyme and the inhibitor. The polymer interferes with the engagement between the inhibitor and the enzyme when a nucleic acid contacts the polymer. In various embodiments, the nucleobase polymer comprises a sequence complementary to that of a target nucleic acid.

In various embodiments, a method of detecting a nucleic acid in a sample is disclosed. The method can comprise combining the sample and a reporter molecule described supra in a mixture, and determining activity of the enzyme in the mixture. An increase in enzyme activity in the sample (compared to a control sample not comprising the target nucleic acid) indicates the presence of the target nucleic acid in the sample.

In some embodiments, a method of making a reporter molecule described above is disclosed. The method can comprise covalently attaching both an enzyme having a k_{cat} of at least about 200 sec⁻¹ and an inhibitor of the enzyme to a nucleobase polymer, wherein upon forming the reporter molecule, the inhibitor can be engaged to the enzyme inhibitorily and wherein a nucleic acid can interfere with the engagement of the inhibitor and the enzyme upon contacting the polymer.

In various embodiments, a kit comprising a reporter molecule described above is disclosed. In certain embodiments, the reporter molecule can be packaged in a container. In certain embodiments, the kit can further comprise a substrate for the enzyme comprised by the reporter molecule.

In various embodiments, a kit for making a reporter molecule described supra is disclosed. In these embodiments, the kit can comprise an enzyme having a k_{cat} of at least about 200 sec⁻¹, and an inhibitor for the enzyme. In certain embodiments, the kit further comprises instructions for covalently attaching the enzyme and the inhibitor to a nucleobase polymer. In certain embodiments, the kit can further comprise a substrate for the enzyme.

### DETAILED DESCRIPTION OF THE EMBODIMENTS

Methods, compositions and kits for detecting nucleic acids are described. The methods and compositions described herein utilize laboratory techniques well known to skilled artisans and can be found in laboratory manuals such as Sambrook, J., Fritsch, E. F. and Maniatis, T. (1989) Molecular Cloning: A Laboratory Manual, 2nd ed. Cold Spring Harbor Laboratory Press, Cold Spring Harbor, N.Y.

A nucleic acid to be detected (herein referred to as a “target nucleic acid”) can be a naturally occurring or synthetic nucleic acid, and in certain embodiments can comprise either DNA or RNA. In some embodiments, the target nucleic acid can be a single-stranded or a double-stranded nucleic acid. If double-stranded, the target nucleic acid can be denatured using techniques well known to skilled artisans.

In various embodiments, a target nucleic acid can comprise at least about ten contiguous nucleotides or at least about ten contiguous base pairs. In certain aspects of the present invention, the reporter molecule can detect short target nucleic acids that are difficult to detect by commonly used methods such as Northern blot hybridization assays and nuclease-based assays. Thus, for example, the reporter molecule can detect nucleic acids having not more than about 40 contiguous bases, not more than about 30 contiguous bases or not more than about 20 contiguous bases.

In certain embodiments, a target nucleic acid can be an RNA that comprises a sequence of at least about 20 contiguous bases to about 25 contiguous bases. Such short RNAs are difficult to convert to cDNAs, and are thus not readily amenable to detection with assays that utilize a deoxyribonuclease. For example, a target RNA molecule can be a microRNA (miRNA) i.e, a regulatory RNA of about 22 nucleotides in length (Ambros, V., Cell 107: 823-826, 2001; Carrington, J. C., and Ambros V., Science 301: 336-338, 2003; Reinhart, B. J., et al., Genes Dev. 16:1616-1626, 2002) or a short interfering RNA (siRNA) i.e., an RNA of approximately 21-25 nucleotides that functions as a sequence-specific mediator of RNA interference in animal cells and post-transcriptional gene silencing in plant cells (Caplen et al., Proc. Nat'l. Acad. Sci. USA 98:9742-9747, 2001; Elbashir et al., EMBO J. 20:6877-6888, 2001; Dykxhoorn, D. M., et al., Nature Reviews Molecular Cell Biology 4:457-467, 2003). Thus, a reporter molecule described herein comprising a nucleobase polymer that is complementary to the entire length of an RNA such as an miRNA or an siRNA can provide a probe of high sensitivity and specificity for detecting a short RNA such as an miRNA or an siRNA. Some other non-limiting examples of target nucleic acids include a gene, an mRNA, a cDNA, a plasmid, a viral nucleic acid, a viroid, a bacteriophage nucleic acid, an exon, an intron, or portions thereof.

In various embodiments, a reporter molecule can comprise an enzyme, an enzyme inhibitor, and a nucleobase polymer extending between the enzyme and the inhibitor. It will be understood by skilled artisans that the term “enzyme” can describe an enzyme moiety of the reporter molecule; the term “enzyme inhibitor” can describe an enzyme inhibitor moiety of the reporter molecule; and the term “nucleobase polymer” can describe a nucleobase polymer moiety of the reporter molecule.

The enzyme of the reporter molecule can comprise an enzyme having a k_{cat} of at least about 200 sec⁻¹ In various embodiments, the enzyme can have a k_{cat} of at least about 300 sec⁻¹. An inhibitor can comprise a reversible inhibitor of the enzyme. A nucleobase polymer moiety can comprise a molecular tether that extends at least between the enzyme moiety and the inhibitor moiety. The nucleobase polymer can comprise a sequence complementary to at least a portion of a target nucleic acid. The sequence complementary to at least a portion of the target nucleic acid can comprise at least about ten nucleotides. In the absence of a target nucleic acid, the inhibitor inhibitorily engages the enzyme. However, when a target nucleic acid contacts the reporter molecule, the nucleobase polymer interferes with the engagement between the inhibitor and the enzyme, thereby increasing enzyme activity. The interaction between the reporter molecule and the target can be of high specificity. In some embodiments, stringency conditions can be selected using methods well known to skilled artisans such that the nucleobase polymer and the target must be at least 70% complementary,at least 80% complementary, at least 90% complementary, at least 95% complementary, or 100% complementary for the nucleobase to interfere with the inhibitory engagement of the inhibitor with the enzyme. Without being limited by theory, it is believed that the nucleobase polymer, if single stranded, is highly flexible and does not significantly interfere with inhibitory engagement of the inhibitor and the enzyme. For example, although covalently attached to the enzyme, the nucleobase polymer is sufficiently flexible such that it can fold or loop back toward the enzyme, allowing the inhibitor to inhibitorily engage the enzyme. However, upon contact between the target nucleic acid and the reporter molecule, the target nucleic acid and the nucleobase polymer form a double-stranded structure comprising base-paired nucleobases. Because it is believed to be less flexible than the single-stranded nucleobase polymer, the more rigid double-stranded structure is less able to fold or loop back, and thereby interferes with the inhibitory engagement of the enzyme by the inhibitor. As a result, enzyme activity is believed to increase when the nucleobase polymer is base-paired with the target nucleic acid (Saghatelian et al., Journal of the American Chemical Society 125:344-345, 2003). Thus, in the absence of the reporter molecule's target nucleic acid, the enzyme moiety of the reporter molecule exhibits a k_{cat} at least two-fold lower than that of the enzyme moiety of the reporter molecule in the presence of the target nucleic acid. In certain embodiments, the enzyme moiety of the reporter molecule can exhibit a k_{cat} at least ten-fold lower than, at least 100-fold lower than, or at least 1000-fold lower than that of the enzyme moiety of the reporter molecule in the presence of its target nucleic acid.

In various embodiments, the enzyme moiety of the reporter molecule can be any enzyme that has a k_{cat} of at least about 200 sec⁻¹. Alternatively, in various embodiments, the enzyme can have a k_{cat} of at least about 300 sec⁻¹. In some embodiments, enzyme activity can be assayed using routine laboratory techniques. An “enzyme” as used herein includes both naturally occurring enzymes as well as variants thereof that retain the same substrate specificity, for example a genetically engineered variant of an enzyme that comprises one or more amino acid changes from a naturally-occurring form of the enzyme, yet remains reactive with the same substrates. Thus, an enzyme can be, in non-limiting example, a naturally occurring enzyme isolated from an naturally-occurring organism, a genetically engineered enzyme expressed by a recombinant organism, or an enzyme that has been chemically modified. A genetic or chemical modification can be any genetic or chemical modification that does not lead to the k_{cat} of the enzyme dropping below about 200 sec⁻¹. A genetic or chemical modification can also be a genetic or chemical modification that does not lead to the k_{cat} of the enzyme dropping below about 300 sec⁻¹. Non-limiting examples of enzyme modifications include alteration, removal, or addition of one or more amino acids to a naturally occurring enzyme, for example one or more amino acid changes resulting from modification of one or more codons comprising a cDNA encoding an enzyme. In non-limiting example, an enzyme modification can comprise a nucleobase polymer attached to an amino acid moiety or a carbohydrate moiety comprised by an enzyme. In another non-limiting example, the enzyme modification can comprise a linker moiety that comprises a covalent bond formed between a cysteine and a sulhydryl-reactive moiety.

Non-limiting examples of enzymes include alkaline phosphatase, β-galactosidase, chloramphenicol acetyl transferase, β-glucuronidase, renilla luciferase, firefly luciferase, and horseradish peroxidase. Non-limiting examples of an alkaline phosphatase that can be used in a reporter molecule include bacterial alkaline phosphatase, shrimp alkaline phosphatase and mammalian alkaline phosphatase. Non-limiting examples of a mammalian alkaline phosphatase can include placental alkaline phosphatase, intestinal alkaline phosphatase and tissue non-specific alkaline phosphatase. In some embodiments, the placental alkaline phosphatase can be a human placental alkaline phosphatase or a secreted human placental alkaline phosphatase (SEAP; Tate, S, S., et al., FASEB J. 4:227-231, 1990).

In various embodiments, the reporter molecule comprises an inhibitor of an enzyme. The inhibitor, in various aspects of these embodiments, can be a reversible inhibitor of the enzyme. The inhibitor can be a covalent inhibitor or a non-covalent inhibitor. The inhibitor of the enzyme can be a competitive inhibitor or a non-competitive inhibitor. The inhibitor of the enzyme can be a transition state mimetic of a substrate of the enzyme. The inhibitor can be a moiety of the reporter molecule and can be covalently attached to the nucleobase polymer. In various embodiments, non-limiting examples of an inhibitor include an alkaline phosphatase inhibitor such as, for example, a phosphate, a phosphonic acid, a thiophosphate, a vanadate, an arsenate, L-phenylalanine, L-homoarginine, L-phenylalanine, levamisole, tetramisole, bromotetramisole, okadaic acid, and theophylline. In various embodiments, non-limiting examples of a phosphonic acid can be phosphonoacetic acid, mercaptomethylphosphonic acid, histidyldiazobenzylphosphonic acid, 2-amino-3-(hydroxy-3-[4-phosphonomethyl-phenylazo])phenyl propionic acid, 3-aminobenzyl phosphonic acid, phenylene-1,3-diphosphonic acid and 2,6-dinitrophenylphosphonic acid. In various embodiments, non-limiting examples of a vanadate can be a (2,2′-bipyridine)oxodiperoxovanadate, an oxodiperoxo-(1,10phenanthroline)vanadate, a picolinato-oxodiperoxo-vanadate, and an oxalato-oxodiperoxovanadate.

In various embodiments, a nucleobase polymer comprised by the reporter molecule can be, in the absence of a target nucleic acid, a single-stranded nucleobase polymer. In various embodiments, a nucleobase polymer can be, for example, an RNA, a DNA, a peptide nucleic acid, a 2′-O-Methyl oligoribonucleic acid, or a locked nucleic acid. In various embodiments, a nucleobase polymer can comprise at least about ten bases. In various embodiments, the bases can comprise a sequence at least about 80% complementary to a contiguous portion of the target nucleic acid. In various embodiments, the bases can comprise a sequence 100% complementary to a contiguous portion of the target nucleic acid. In various configurations, the nucleobase polymer can comprise a sequence of at least about 20 contiguous bases to about 24 contiguous bases.

In various embodiments, the nucleobase polymer can be at least about 20 bases in length, or at least about 25 bases in length. However, in some embodiments, for the nucleobase to interfere with the engagement between the enzyme and the inhibitor, no more than about 10, no more than about 15, or no more than about 20 nucleotides can remain unpaired with a target nucleic acid. In some embodiments, the nucleobase polymer is less than about 80 bases in length, less than about 40 bases in length, or less than about 30 bases in length.

In various embodiments, enzyme activity of the reporter molecule can be detected using an enzyme substrate. An enzyme substrate can be, for example, a chromogenic substrate, a fluorogenic substrate, a radioactive substrate or a chemiluminescent substrate. In non-limiting example, a chemiluminescent substrate can comprise a 1,2-dioxetane moiety. In various embodiments, a reporter molecule can comprise an alkaline phosphatase moiety and a substrate can be a chemiluminescent substrate such as a 3-(4-methoxyspiro [1,2-dioxetane-3,2′(5′-chloro)-tricyclo [3.3.1.1^{3,7}]decan]-4-yl)phenylphosphate. In various embodiments, a reporter molecule can comprise a β-galactosidase and the chemiluminescent substrate can comprise a 1,2-dioxetane moiety such as a 3-(4-methoxyspiro-[1,2-dioxetane-3,2′tricyclo-[3.3.1.1 ^{3,7}]decan]-4-yl)phenyl-β-D-galactopyranoside.

In various embodiments, a kit can comprise the reporter molecule is described supra. A kit comprising the reporter molecule can further comprise instructions. A kit comprising the reporter molecule can further comprise packaging.

In various embodiments, a kit comprising the reporter molecule can further comprise a substrate for the enzyme comprised by the reporter molecule. The substrate can be any substrate that can yield a detectable reaction product upon reaction with the enzyme. In non-limiting example, a substrate for the enzyme comprised by a reporter molecule of a kit can be a chromogenic substrate, a fluorogenic substrate, a radioactive substrate or a chemiluminescent substrate. A chemiluminescent substrate can comprise, in non-limiting example, a 1,2-dioixetane moiety. In various embodiments, a kit can comprise a reporter molecule comprising an alkaline phosphatase moiety, and a chemiluminescent alkaline phosphatase substrate such as, for example, a 3-(4-methoxyspiro [1,2-dioxetane-3,2′(5′-chloro)-tricyclo[3.3.1.1^{3,7}]decan]-4-yl)phenylphosphate. In various embodiments, a kit can comprise a reporter molecule comprising a β-galactosidase moiety, and a chemiluminescent β-galactosidase substrate such as, for example, a 1,2-dioxetane moiety is 3-(4-methoxyspiro-[1,2-dioxetane-3,2′-tricyclo-[3.3.1.1^{3,7}]decan]-4-yl)phenyl-β-D-galactopyranoside.

In various embodiments, a kit for making the reporter molecule described supra is disclosed. The kit can comprise an enzyme having a k_{cat} of at least about 200 sec⁻¹ and an inhibitor for the enzyme. In some embodiments, the enzyme can have a k_{cat} of at least about 300 sec⁻¹. In certain embodiments, the kit can further comprise instructions for covalently attaching the enzyme and the inhibitor to a nucleobase polymer. In certain configurations, the kit can also comprise at least one reagent for covalently attaching the enzyme to the nucleobase polymer. The reagent can be a chemical linker. The reagent can, in some configurations, comprise at least one reactive moiety. Each reactive moiety can be, for example, an amine-reactive moiety, a carboxyl-reactive moiety, a hydroxyl-reactive moiety, or a thiol-reactive moiety. The thiol-reactive moiety can be, for example, a pyridyl disulfide.

In various embodiments, the kit can comprise a reagent for covalently attaching the inhibitor to the nucleobase polymer. The kit, in certain embodiments, can comprise the nucleobase polymer. The nucleobase polymer can comprise at least one reactive moiety, and each reactive moiety can be, for example, an amine-reactive moiety, a carboxyl-reactive moiety, a hydroxyl-reactive moiety, or a thiol-reactive moiety. A thiol-reactive moiety can be, for example, a pyridyl disulfide.

In various embodiments, the kit can comprise an inhibitor comprising an enzyme-inhibitory moiety and a reactive moiety. In certain embodiments, the reactive moiety can be, for example, an amine-reactive moiety, a carboxyl-reactive moiety, a hydroxyl-reactive moiety, and a thiol-reactive moiety. A thiol-reactive moiety can be, for example, a pyridyl disulfide. In certain embodiments, the inhibitor can be a reversible inhibitor, as described supra.

In various embodiments, the kit can be used to construct a reporter molecule as described supra. The enzyme and the inhibitor of the kit can be combined with a nucleobase polymer that comprises a sequence complementary to a target nucleic acid, as described supra. The nucleobase polymer, the inhibitor, and/or the enzyme can comprise a reactive group that can be used for covalently linking the nucleobase polymer to the enzyme and the inhibitor. In certain embodiments, a user can provide a nucleobase polymer, as described supra. The nucleobase polymer can be combined with the enzyme and the inhibitor, and thereby form a reporter molecule that can be used to detect a target nucleic acid comprising a sequence complementary to the nucleobase polymer, as described supra. The nucleobase polymer can be a nucleobase polymer such as, for example, an RNA, a DNA, a peptide nucleic acid, a 2′-O-Methyl oligoribonucleic acid, and a locked nucleic acid, i.e., an oligonucleotide wherein a bicyclic ribofuranosyl nucleotide monomer is linked between the 2′-oxygen and the 4′-carbon atoms by at least one methylene unit (Braasch, D. A., et al., Chem. Biol. 8: 1-7, 2001; Petersen, M., et al., J. Am. Chem. Soc. 124:5974-5982, 2002; PCT applications WO 98/22489 to Takeshi, WO 98/39352 to Satoshi et al., and WO 99/14226 to Jesper et al). A target nucleic acid that can be detected using reporter molecule constructed using the kit can be any nucleic acid comprising at least ten bases or base pairs, such as, in non-limiting example, an miRNA or an siRNA. An enzyme of the kit can be any enzyme having a k_{cat} of at least about 200 sec^{31 1}, such as the enzymes described supra. An enzyme of the kit can also be an enzyme having a k_{cat} of at least about 300 sec⁻¹. Furthermore, the inhibitor can be any inhibitor as disclosed supra.

In various embodiments, the kit can further comprise a substrate for the enzyme, such as a chromogenic substrate, a fluorogenic substrate, a radioactive substrate and a chemiluminescent substrate as described supra.

In various configurations, methods of making a reporter molecule described supra are disclosed. In certain configurations, a method can comprise covalently attaching both an enzyme having a k_{cat} of at least about 200 sec⁻and an inhibitor of the enzyme to a nucleobase polymer, wherein upon forming the reporter molecule, the inhibitor is engaged to the enzyme inhibitorily and wherein the nucleic acid interferes with the engagement of the inhibitor and the enzyme upon contacting the polymer. In certain configurations, the enzyme can have a k_{cat} of at least about 300 sec⁻¹. In certain configurations, The nucleobase polymer can be attached to the enzyme at any available site, wherein attachment of the polymer does not reduce the enzyme's k_{cat} below about 200 sec⁻¹. In some configurations, attachment of the polymer to the enzyme does not reduce the enzyme's k_{cat} below about 300 sec⁻¹. In certain embodiments, attaching the nucleobase polymer to the enzyme can comprise reacting the enzyme and/or the nucleobase polymer with at least one chemical linker that is reactive covalently towards the enzyme and/or the nucleobase polymer. The nucleobase polymer can be attached to the inhibitor at any available site, wherein attaching the polymer to the inhibitor does not destroy the inhibitor's ability to inhibit the enzyme (in the absence of a target nucleic acid).

In various embodiments, a method is described for detecting a nucleic acid such as a target nucleic acid in a sample. The method, in certain embodiments, comprises combining the sample and a reporter molecule as described supra in a mixture, and determining enzyme activity in the mixture. The nucleic acid can be any nucleic acid target, such as those described supra. In certain configurations, the method can comprise combining in a mixture a reporter molecule, a target nucleic acid, and substrate for the enzyme comprised by the reporter molecule, and determining enzyme activity in the mixture. Determining enzyme activity in the mixture can comprise, for example, measuring the rate of formation of a reaction product resulting from contact between the enzyme and the enzyme substrate. Standard methods known to skilled artisans can be used to determine enzyme activity. For example, the substrate can be a chemiluminescent substrate for the enzyme comprising the reporter molecule, and a standard method for detecting photonic emission, such as exposing the mixture to a light-sensitive emulsion (for example, an emulsion comprised by an X-ray film) or a photon counter can be used to determine enzyme activity. In another example, the substrate can be a fluorogenic substrate for the enzyme comprising the reporter molecule, and a standard method for detecting fluorescent light emission can be used to determine enzyme activity.

### EXAMPLE 1

This example illustrates a method that can be used for making a reporter molecule for detecting a nucleic acid.

In this example, recombinant human placental alkaline phosphatase (PLAP) can be obtained using techniques known in the art (Berger J., et al., Proc. Nat'l. Acad. Sci. USA 84:4885-4889, 1987). Mammalian alkaline phosphatases comprise a single free cysteine residue (Cys-101) that can be derivatized without significantly diminishing the k_{cat} of the enzyme (Kozlenkov, A., et al., J. Biol. Chem. 277:22992-22999, 2002). Because of the availability of Cys-101 for derivatization, a nucleobase polymer comprising an RNA sequence complementary to a 22-nucleotide siRNA (Elbashir SM, et al., Genes Dev. 15:188-200, 2001) and a thiol moiety can be synthesized using solid-phase synthesis, activated as a pyridyl disulfide, and linked to Cys-101 of PLAP (Saghatelian A., et al., supra; Kozlenkov et al., supra). In addition, an alkaline phosphatase inhibitor comprising a phosphonic acid can be also attached to the nucleobase polymer (Davini, E., et al., Genet Anal. Tech. Appl. 9:39-47,1992).

### EXAMPLE 2

This example illustrates how the reporter molecule of the present invention can be used to detect an siRNA sequence.

A mixture can be formed of the reporter molecule as described in Example 1 and an RNA extract from Drosophila cells (Elbashir S M, et al., Genes Dev. 15:188-200, 2001). The chemiluminescent alkaline phosphatase substrate 3-(4-methoxyspiro [1,2-dioxetane-3,2′(5′-chloro)-tricyclo[3.3.1.13, 7]decan]-4-yl)phenyphosphate can also added to the mixture. A photon detector can then used to measure light emission from the mixture. An increase in light emission from the sample compared to a control lacking the extract indicates the presence of the siRNA.

### EXAMPLE 3

This example illustrates how the reporter molecule of the present invention can be used in a diagnostic test for detection of the RNA sequences in a human tissue. Reporter molecules, each comprising a different nucleobase polymer complementary to transcripts known to vary with a disease state, can be distributed to identified loci in a microarray. The chemiluminescent alkaline phosphatase substrate 3-(4-methoxyspiro [1,2-dioxetane-3,2′(5′-chloro)-tricyclo [3.3.1.13, 7]decan]-4-yl)phenylphosphate can be added to a cell extract from a tissue sample of a patient, forming a mixture. Aliquots of the mixture can then be added to each locus on the microarray. Light emission from each locus can be then measured using a microarray reader, and recorded in a digital computer. Transcript levels can then be compared to transcript levels from healthy tissue to aid in disease diagnosis.

As various changes could be made in the above methods and compositions without departing from the scope of the invention, it is intended that all matter contained in the above description be interpreted as illustrative and not in a limiting sense.

All references cited in this specification are hereby incorporated by reference in their entirety. The discussion of the references herein is intended merely to summarize the assertions made by their authors and no admission is made that any reference constitutes prior art relevant to patentability. Applicants reserve the right to challenge the accuracy and pertinency of the cited references.

1. A reporter molecule for detecting a nucleic acid, the molecule comprising:
an enzyme having a k_{cat} of at least about 200 sec⁻¹;
a reversible inhibitor of said enzyme inhibitorily engaging said enzyme; and
a nucleobase polymer extending between said enzyme and said reversible inhibitor;
wherein said polymer is operable to interfere with the engagement between said inhibitor and said enzyme when said nucleic acid contacts said polymer.

2. A reporter molecule according to claim 1 wherein said nucleic acid is selected from the group consisting of a miRNA and a siRNA.

3. A reporter molecule according to claim 1 wherein said enzyme is selected from the group consisting of alkaline phosphatase, β-galactosidase, chloramphenicol acetyl transferase, β-glucuronidase, renilla luciferase, firefly luciferase, and horseradish peroxidase.

4. A reporter molecule according to claim 3 wherein said enzyme is an alkaline phosphatase selected from the group consisting of bacterial alkaline phosphatase, shrimp alkaline phosphatase and a mammalian alkaline phosphatase.

5. A reporter molecule according to claim 1 wherein said reversible inhibitor of the enzyme is a transition state mimetic of a substrate of the enzyme.

6. A reporter molecule according to claim 1 wherein said reversible inhibitor of the enzyme is selected from the group consisting of phosphate, phosphonic acid, thiophosphate, vanadate, arsenate, L-phenylalanine, L- homoarginine, L-phenylalanine, levamisole, tetramisole, bromotetramisole, okadaic acid, theophylline, and mixtures thereof.

7. A reporter molecule according to claim 1 wherein said nucleobase polymer is selected from the group consisting of RNA, DNA, peptide nucleic acid, a 2′-O-Methyl oligoribonucleic acid, and locked nucleic acid.

8. A reporter molecule according to claim 1 wherein said nucleobase polymer comprises at least about 10 bases, said 10 bases comprising a sequence at least about 80% complementary to a contiguous portion of said nucleic acid.

9. A reporter molecule according to claim 1 wherein said nucleobase polymer comprises at least about 10 bases, said 10 bases comprising a sequence about 100% complementary to a contiguous portion of said nucleic acid.

10. A reporter molecule according to claim 1 wherein said nucleobase polymer comprises a sequence of from about 20 to about 24 contiguous bases.

11. A method of detecting a nucleic acid in a sample, the method comprising:
contacting said sample with a reporter molecule for detecting said nucleic acid, wherein said reporter molecule comprises an enzyme having a k_{cat} of at least about 200 sec⁻¹, a reversible inhibitor of said enzyme inhibitorily engaging said enzyme; and a nucleobase polymer extending between said enzyme and said reversible inhibitor, said polymer operable to interfere with the engagement between said inhibitor and said enzyme when said nucleic acid contacts said polymer; and
determining activity of said enzyme.

12. A method according to claim 11 wherein said nucleic acid is selected from the group consisting of a miRNA and a siRNA.

13. A method according to claim 11 wherein said enzyme is selected from the group consisting of an alkaline phosphatase, a β-galactosidase, a chloramphenicol acetyl transferase, a β-glucuronidase, a renilla luciferase, a firefly luciferase, and a horseradish peroxidase.

14. A method according to claim 13 wherein said enzyme is an alkaline phosphatase selected from the group consisting of a bacterial alkaline phosphatase, a shrimp alkaline phosphatase and a mammalian alkaline phosphatase.

15. A method according to claim 11 wherein said reversible inhibitor of the enzyme is selected from the group consisting of phosphate, phosphonic acid, thiophosphate, vanadate, arsenate, L-phenylalanine, L-homoarginine, L-phenylalanine, levamisole, tetramisole, bromotetramisole, okadaic acid, and theophylline.

16. A method according to claim 11 wherein said nucleobase polymer is selected from the group consisting of RNA, DNA, peptide nucleic acid, 2′-O-Methyl oligoribonucleic acid, and locked nucleic acid.

17. A method according to claim 11 wherein said nucleobase polymer comprises a sequence of from about 20 to about 24 contiguous bases.

18. A method according to claim 11, further comprising contacting the reporter molecule with a substrate for said enzyme.

19. A method according to claim 18 wherein said substrate is selected from the group consisting of chromogenic substrate, fluorogenic substrate, radioactive substrate and chemiluminescent substrate.

20. A method of making a reporter molecule for detecting a nucleic acid comprising:
covalently attaching both an enzyme having a k_{cat} of at least about 200 sec⁻¹ and a reversible inhibitor of said enzyme to a nucleobase polymer, wherein upon forming said reporter molecule, said reversible inhibitor is engaged to said enzyme inhibitorily and wherein said nucleic acid is operable to interfere with the engagement of said inhibitor and said enzyme upon contacting said polymer.

21. A method according to claim 20 wherein the nucleic acid is selected from the group consisting of a miRNA and a siRNA.

22. A method according to claim 20 wherein said enzyme is selected from the group consisting of alkaline phosphatase, β-galactosidase, chloramphenicol acetyl transferase, β-glucuronidase, renilla luciferase, firefly luciferase, and horseradish peroxidase.

23. A method according to claim 20 wherein said enzyme is an alkaline phosphatase selected from the group consisting of bacterial alkaline phosphatase, shrimp alkaline phosphatase and mammalian alkaline phosphatase.

24. A method according to claim 20 wherein said reversible inhibitor of the enzyme is selected from the group consisting of phosphate, phosphonic acid, thiophosphate, vanadate, arsenate, L-phenylalanine, L-homoarginine, L-phenylalanine, levamisole, tetramisole, bromotetramisole, okadaic acid, and theophylline.

25. A method according to claim 20 wherein said nucleobase polymer is selected from the group consisting of RNA, DNA, peptide nucleic acid, 2′-O-Methyl oligoribonucleic acid, and a locked nucleic acid.

26. A method according to claim 20 wherein said nucleobase polymer comprises a sequence comprising at least about 10 bases, the sequence at least about 80% complementary to a contiguous portion of the nucleic acid.

27. A method according to claim 20 wherein said nucleobase polymer comprises a sequence comprising from about 20 to about 24 contiguous bases.

28. A method according to claim 20, further comprising linking said nucleobase polymer and said enzyme with a chemical linker.

29. A method according to claim 28 wherein said chemical linker comprises at least two reactive moieties, wherein each reactive moiety is independently selected from the group consisting of an amine-reactive moiety, a carboxyl-reactive moiety, a hydroxyl-reactive moiety, and a thiol-reactive moiety.

30. A method according to claim 20, further comprising reacting said enzyme with a chemical precursor of the polymer comprising a protein-reactive moiety.

31. A method according to claim 30 wherein said protein-reactive moiety is selected from the group consisting of an amine-reactive moiety, a carboxyl-reactive moiety, a hydroxyl-reactive moiety, and a thiol-reactive moiety.

32. A method for detecting a small RNA, said method comprising:
providing an enzyme having a k_{cat} of at least about 200 sec⁻¹;
tethering a reversible inhibitor enzyme to said small RNA;
hybridizing said small RNA with a complementary nucleotide;
determining enzyme activity produced by said hybridizing said small RNA with said complementary nucleotide; and
relating determined enzyme activity to a quantity of said small RNA.

33. A method according to claim 32, further comprising contacting said enzyme to a substrate.

34. A method according to claim 33, further comprising cleaving said enzyme from said substrate during said hybridizing said small RNA with a complementary nucleotide.

35. A method according to claim 34, further comprising producing a fluorescent or a chemiluminescent signal from said cleaving said enzyme from said substrate.

36. A method according to claim 32 wherein said small RNA is selected from the group comprising siRNA and miRNA.

37. A system for detecting a nucleic acid, said system comprising:
a reporter molecule for detecting said nucleic acid, wherein said reporter molecule comprises an enzyme having a k_{cat} of at least about 200 sec⁻¹, a reversible inhibitor of said enzyme inhibitorily engaging said enzyme; and a nucleobase polymer extending between said enzyme and said reversible inhibitor, said polymer operable to interfere with the engagement between said inhibitor and said enzyme when said nucleic acid contacts said polymer; and
a substrate for said enzyme.

38. A system according to claim 37 wherein said enzyme is selected from the group consisting of alkaline phosphatase, β-galactosidase, chloramphenicol acetyl transferase, β-glucuronidase, renilla luciferase, firefly luciferase, and horseradish peroxidase.

39. A system according to claim 37 wherein said reversible inhibitor of the enzyme is selected from the group consisting of phosphate, phosphonic acid, thiophosphate, vanadate, arsenate, L-phenylalanine, L-homoarginine, L-phenylalanine, levamisole, tetramisole, bromotetramisole, okadaic acid, and theophylline.

40. A system according to claim 37 wherein said nucleobase polymer is selected from the group consisting of RNA, a DNA, peptide nucleic acid, 2′-O-Methyl oligoribonucleic acid, and locked nucleic acid.

41. A method according to claim 37, wherein said substrate emits a signal which changes depending on whether the reporter molecule contacts said nucleic acid

42. A method according to claim 41 wherein said substrate is selected from the group consisting of chromogenic substrate, fluorogenic substrate, radioactive substrate and chemiluminescent substrate.

43. A system according to claim 41, further comprising a detection system detecting a signal from said enzyme.

44. A system according to claim 43, further comprising a microprocessor collecting and analyzing said signal.

45. A reporter molecule for detecting a nucleic acid, the molecule comprising:
an enzyme moiety having a k_{cat} of at least about 200 sec⁻¹;
a reversible inhibitor moiety, operable as a reversible inhibitor of said enzyme; and
a nucleobase polymer moiety having at least about 20 bases, covalently bonded to said enzyme moiety and said reversible inhibitor moiety;
wherein the said polymer is operable to allow said inhibitor moiety to reversibly inhibit said enzyme moiety, and to interferes with such inhibition when said nucleic acid contacts said polymer.

46. A reporter molecule according to claim 45 wherein said enzyme is selected from the group consisting of alkaline phosphatase, β-galactosidase, chloramphenicol acetyl transferase, β-glucuronidase, renilla luciferase, firefly luciferase, and horseradish peroxidase.

47. A reporter molecule according to claim 45 wherein said enzyme is an alkaline phosphatase selected from the group consisting of bacterial alkaline phosphatase, shrimp alkaline phosphatase and a mammalian alkaline phosphatase.

48. A reporter molecule according to claim 45 wherein said reversible inhibitor of the enzyme is selected from the group consisting of phosphate, phosphonic acid, thiophosphate, vanadate, arsenate, L-phenylalanine, L-homoarginine, L-phenylalanine, levamisole, tetramisole, bromotetramisole, okadaic acid, theophylline, and mixtures thereof.

49. A reporter molecule according to claim 45 wherein said nucleobase polymer is selected from the group consisting of RNA, DNA, peptide nucleic acid, a 2′-O-Methyl oligoribonucleic acid, and locked nucleic acid.

50. A reporter molecule according to claim 45 wherein said nucleobase polymer comprises at least about 10 bases having a sequence at least about 80% complementary to a contiguous portion of said nucleic acid.

51. A reporter molecule according to claim 45 wherein said nucleobase polymer comprises a sequence of from about 20 to about 40 contiguous bases.
